(19) United States
(12) Patent Application Publication
(10) Pub. No.: US 2021/0324330 A1
(43) Pub. Date: Oct. 21, 2021

(54) METHODS FOR NONINVASIVE DETECTION, DIAGNOSIS AND TREATMENT OF DISEASE

(71) Applicant: Noninvasive Technologies, LLC, Elkridge, MD (US)

(72) Inventor: Padmanabhan P. NAIR, Ellicott City, MD (US)

(73) Assignee: Noninvasive Technologies, LLC, Elkridge, MD (US)

(21) Appl. No.: 17/230,435

(22) Filed: Apr. 14, 2021

Related U.S. Application Data
(60) Provisional application No. 63/010,778, filed on Apr. 16, 2020.

(51) Int. Cl. C12N 5/071; G01N 33/569; C07K 16/10; G01N 33/574; C07K 16/30; C07K 16/12

(52) U.S. Cl. CPC C12N 5/068; G01N 33/56911; C07K 16/1045; C07K 16/12; G01N 33/57484; C07K 16/30; G01N 33/56983

(57) ABSTRACT

The methods of the present invention include noninvasive methods for the isolation of antibody-producing cells from the gut and the use of the cells so-isolated to generate antibodies responsive to biomarkers for a number of health conditions. The responsive antibodies are chimeric secretory antibodies comprising IgA and IgG moieties that may be useful in the diagnosis and treatment of various health conditions after challenged with biomarkers thereof. In a preferred embodiment, stool samples may be obtained from patients suffering from HIV infection and cells may be isolated from the samples according to the methods described herein and reacted with antibodies responsive to HIV biomarkers. The diagnostic methods described herein allow for room temperature sample isolation for up to five days prior to diagnosis and are useful in detecting latent HIV that cannot be detected in blood samples. It is another object of the invention to generate therapeutic antibodies.

### CROSS-REFERENCE TO RELATED APPLICATIONS

This application claims the benefit of the priority date of U.S. Provisional Patent Application Ser. No. 63/010,778, filed Apr. 16, 2020, the contents of which are hereby incorporated in their entirety.

### STATEMENT REGARDING FEDERALLY SPONSORED RESEARCH OR DEVELOPMENT

The invention described herein was conceived and reduced to practice without the benefit of federal funding.

### BACKGROUND OF THE INVENTION

The invention according to the embodiments described herein relates broadly to the fields of cell biology, microbiology and immunology, and more specifically to the field of coprocytobiology.

Somatic cell sampling and recovery (SCSR) is a technique that was developed by the applicant to isolate viable gut cells, especially of colonic origin, from stool samples obtained from human and animal patients. SCSR has since been used by the applicant to elucidate a great deal about the exfoliated cell populations present in the stool, primarily of colonic origin. The details of SCSR and what the applicant has learned about the exfoliated viable colonic cells isolated via SCSR are described in some detail in the article, Coprocytobiology On the Nature of Cellular Elements from Stools in the Pathophysiology of Colonic Disease, J. Clin. Gastroenterol., 2003; 36(Suppl. 1):S84-93, the contents of which are incorporated herein by reference in their entirety.

The study of exfoliated colonocytes obtained through SCSR has given rise to many important research findings related to specific disease progressions, such as those associated with inflammatory bowel disease (IBD) and colon cancer. It is an object of the present invention to leverage the technology described herein to improve methods for the diagnosis and surveillance of the progressions of diseases affecting not only the gut, but other regions of the human and animal anatomies.

For example, human immunodeficiency virus (HIV) infection is a global condition that affects more than 75 million people. Current observations suggest that the virus preferentially attacks immune cells in the digestive tract rather than in the blood. Monitoring the so-called “latent HIV” in the gut tissue during treatment is a far more sensitive methodology for following disease progression than is measuring viral load or counting lymphocytes in the blood expressing cluster of differentiation 4 (CD4). Other immunodeficiency diseases may be diagnosed more rapidly and noninvasively using the methods described herein, as will become clear according to the present disclosure.

In recent years, we have been seized with the conundrum of latent HIV where the virus appears to be sequestered in the digestive tract with no immunological response detectable in the blood. This was found by the applicant to be the result of a total absence of a bi-specific antibody (a heterodimer of IgA/IgG) produced in the gut. Moreover, the applicant discovered in a small cohort study that subjects with this deficiency tend to be of African-American lineage. Subsequent studies led to the conclusion that this is a new genetic defect involving the germ-line deletion of the bi-specific antibody sequence (a syndrome similar to sickle-cell anemia). The applicant inferred from this that perhaps HIV is able to reside in the digestive tract undisturbed because of this defect in the gut cells.

Currently, there are no readily available noninvasive methods of obtaining gut cells. Current methods involve invasive gastrointestinal biopsies. These procedures involve the use of a colonoscope on the patient as well as the participation of a physician (endoscopist) in a hospital or other medical institution, all of which are available only at significant cost to the healthcare system. Therefore, it is one object of the invention of the present disclosure to provide a less expensive and less invasive alternative by leveraging SCSR to obtain gut cells for analysis, and also for culture and production of therapeutic antibodies as described below.

### SUMMARY OF THE INVENTION

The methods described in the present disclosure allow clinicians to isolate gut cells noninvasively from a small stool samples at room temperature and preserve them at room temperature for testing for the presence of disease conditions such as latent HIV in subjects from which the samples are isolated. The methods described herein create great potential for augmenting the current global effort devoted to controlling HIV infection and provide a rational approach to monitoring disease progression during treatment.

Briefly, a small stool sample about the size of a pea is collected in SCSR transport medium, which is a special liquid medium that allows for storage and shipment at room temperature without compromising the sample. The cells in the sample will remain alive up to nine days at room temperature in the SCSR transport medium, during which time they can be isolated for culture as well as detection and quantification of disease markers such as those indicative of the presence of HIV in the gut cells using techniques that will be familiar to one of ordinary skill in the art.

In addition to providing noninvasive methodologies for the detection of disease markers, gut cells isolated according to methods of the present disclosure may comprise the isolation of gastrointestinal progenitor stem cells, referred to herein as “GIPC” cells. The applicant has observed that GIPC cells may comprise membrane bound tumor antibodies. It is a further object of the invention to provide for a method of partial differentiation of GIPC cells toward plasma-like progeny that exhibit B-lymphocyte behavior. In particular, the techniques of the invention may be utilized to convert membrane-bound GIPC antibodies to chimeric secretory antibodies comprising immunoglobulin A and G (IgA and IgG) moieties capable of challenge with any number of antigens using techniques familiar to one of ordinary skill in the art. In this manner, chimeric secretory antibodies with anti-tumor or anti-infective properties may be raised much more efficiently than possible using conventional methods such as hybridomas. Moreover, such antibodies, herein collectively referred to as “GIPSITUMAB”, are broad spectrum and may prove more effective, for example, in the detection of HIV, which is capable of evading detection by mutating and rendering capsid proteins ineffective as biomarkers of the disease. These and other benefits of the present invention will be clear to one of ordinary skill in the art from the disclosure that follows.

### BRIEF DESCRIPTION OF THE DRAWINGS

FIG. 1 illustrates the steps of a method for isolating gut cells from a stool sample according to the present disclosure.

FIG. 2 is a flow chart illustrating the various useful methods that may be carried out following gut cell isolation according to FIG. 1.

FIG. 3 illustrates the sequence of appearance of laboratory biomarkers for HIV-1 infection.

### DETAILED DESCRIPTION OF THE INVENTION

In certain embodiments, the invention of the present disclosure comprises a multi-step method or process for noninvasively obtaining cells from within the gastrointestinal tract of a human or animal patient by first obtaining a stool sample, then storing the sample in a transport medium that allows living cells within the sample to survive for several days prior to isolation for subsequent study and propagation. In certain embodiments, cells so isolated may comprise progenitor cells (including GIPC cells), epithelial cells, leukocytes and combinations thereof. These cell populations may further comprise membrane-bound antibodies responsive to tumor or other antigens or biomarkers for various disease states.

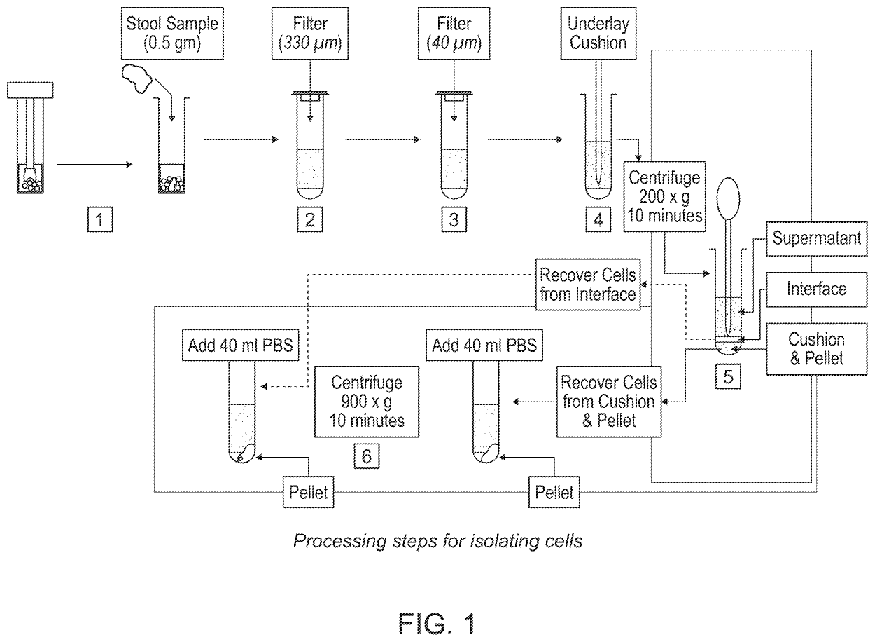

For example, FIG. 1 illustrates method steps for isolating cells for later use according to the present disclosure. The method begins by obtaining a small stool sample (e.g., 0.5 grams) 1; filtering to remove particulate matter 330 microns in diameter or greater 2; further filtering to remove particles greater that 40 microns in diameter 3; providing an cushioning underlay 4; centrifuging at 200×g for 10 minutes to isolate a pellet within the cushioning underlay 5 and recovering cells from the cushion and pellet; and optionally isolating the pellet by centrifugation at 900×g for 10 minutes 6.

In certain embodiments, the methods described herein further comprise collecting a small stool sample from a human patient using noninvasive means, then isolating the gut cells from the stool sample and testing for the presence of HIV using techniques that will be familiar to those skilled in the art. Currently, testing of HIV is primarily carried out using blood samples which require patients to have a needle inserted into a blood vessel for blood collection, an invasive and uncomfortable method for obtaining cells from the blood to test for HIV. Even worse, in order to detect latent HIV residing in the gut cells of a patient, the patient must undergo a still more invasive, protracted and painful endoscopy and biopsy of gastrointestinal tissue, which requires costly oversight by a qualified physician and the costly use of hospital facilities.

In preferred embodiments, gut cells comprising GIPC cells may be isolated noninvasively using SCSR and partially differentiated, leading to the conversion of membrane-bound antibodies to GIPSITUMAB, which may then be challenged with biomarkers or antigens of interest in order to isolate GIPSITUMAB useful in diagnosing specific diseases. In certain embodiments, GIPSITUMAB with broad spectrum responsiveness to HIV may be used to detect latent HIV in gut cells isolated using SCSR technology.

The invention of the present disclosure has its roots in the discovery by the applicant that not all fecal cells in the stool are necrotic, and in fact many are alive and can be isolated, cultured and stimulated to express specific genes. In certain embodiments of the invention described herein, SCSR is employed for the purpose of isolating millions of exfoliated host cells per gram of fecal sample. These cells can be stored within a temperature range comprising room temperature for up to five days in SCSR transport medium prior to isolation from the sample. Room temperature for the purposes of the present disclosure is considered to be a temperature range that includes temperatures not less than 68° Fahrenheit and not more than 78° Fahrenheit. Cells are isolated using a simple step gradient centrifugation protocol followed by wash in phosphate buffered saline (PBS) solution or equivalent. The isolated cell populations include all viable cell types, such as epithelial cells, progenitor cells (e.g., GIPC cells) and leukocytes.

The preservation of gut cells using SCSR enables flexible clinical scheduling of cell isolation and investigation of gastrointestinal genetic and cell surface markers without the need for invasive procedures such as gut biopsies. Additionally, cell archives and complimentary deoxyribonucleic acid (cDNA) libraries can be constructed for future study. Cells isolated using a procedure as described herein may be subjected to any number of known analytical techniques such as flow cytometry, and progenitor cell lines be maintained for multiple generations. Cells isolated using SCSR also yield better sample material for the isolation of DNA and conduct of polymerase chain reaction (PCR) amplification for diagnostic methods, as signal degradation due to microbial interference and other complications associated with fecal collection are diminished.

The gut cells isolated using SCSR may be useful for sampling and testing for any number of clinical disease markers, including but not limited to biomarkers for cancer, markers for bacterial and viral infection such as infection by HIV, as well as markers for other conditions such as inflammatory bowel disease. In addition to their diagnostic utility, gut cells isolated using SCSR may be cultured and partially differentiated to produce therapeutic GIPSITUMAB for the treatment of diseases responsive to antibody intervention.

In certain embodiments, SCSR involves collecting a small stool sample, such as less than one gram, in SCSR transport medium under normal ambient temperature or room temperature for cell isolation within five days thereafter. In certain embodiments, a suspension is created and filtered, then under layered with a high-density medium and centrifuged at low speed for about ten minutes. Cells are extracted from the interface between the high-density medium and supernatant. For example, SCSR media containing gut cells may be filtered to exclude particles greater than 330 microns in diameter, then again to exclude particles larger than 40 microns in diameter filter, before centrifugation, for example, at 200 g for 10 minutes and subsequent isolation steps as depicted in FIG. 1.

In certain embodiments, cells isolated from the gut according to a method or process as described herein may express various biomarkers. As used herein, the term “biomarker” may be used to designate a protein, carbohydrate, nucleic acid or any combination thereof or constituent or combination of constituents thereof, the presence or absence of which in a population of cells isolated as described herein indicates or suggests the presence or absence of a pathology or disease condition of a patient from which a stool sample was taken to isolate the cells as described herein.

In one embodiment, a cell population isolated as described herein may also comprise GIPC cells comprising membrane bound anti-tumor or anti-infective antibodies that may be partially differentiated to produce chimeric secretory antibodies that are IgA/IgG heterodimers, the absence of which may indicate or suggest a pathology or disease condition comprising an immunodeficiency syndrome specific to a patient population. In certain embodiments, the IgG moiety binds to a common tumor-associated membrane antigen (MATA) resulting in osmolytic tumor cell death, while the IgA moiety recognizes cells infected by destructive microorganisms such as viruses and bacteria. In one embodiment, a patient population deficient in GIPC cell membrane-bound antibodies as described above comprises a majority of African-American patients, the deficiency being indicative of an immunodeficiency condition unique to African-Americans and possibly increased cancer risk. Further, in certain embodiments a method as described herein further comprises utilizing cells expressing a chimeric IgA/IgG antibody and inducing such cells to produce therapeutic antibodies to viral and bacterial pathogens using known immunological methods. In certain embodiments, certain methods as described herein may be used to produce therapeutic antibodies to pathogens selected from the group consisting of Ebola virus, Marburg virus, HIV, influenza and anthrax, and may be useful in the treatment of individuals deficient in these antibodies.

In one embodiment, a biomarker indicates or suggests the presence of HIV. In one embodiment, the biomarker of HIV is not detectable in blood samples prepared according to current clinical methods comprising the invasive collection of patient blood. In one embodiment, HIV not detectable according to methodologies other than those described herein may be described as “latent” HIV. In one embodiment, a methodology other than one described herein comprises methods employed to detect viral load of HIV in a blood sample obtained from a patient through invasive means. In one embodiment, a population of patients from whom samples are obtained by such invasive means is considered an HIV-negative population based on blood testing comprising such invasive means of sample collection. In this case, an object of a method of latent HIV detection according to the present disclosure comprises identifying HIV-positive patients that have been diagnosed as HIV-negative according to blood sample testing, referred to herein as “false negatives”. Methods according to such embodiments leverage the detectable expression of HIV biomarkers such as viral load according to PCR-based methods or measurement of the number of cells expressing the CD4 biomarker, a decline of which is associated with positive HIV diagnosis. In certain preferred embodiments, detection of HIV biomarkers may be achieved by raising responsive GIPSITUMAB for the purposes of broad-spectrum HIV detection in isolated gut cells according to methods as described herein.

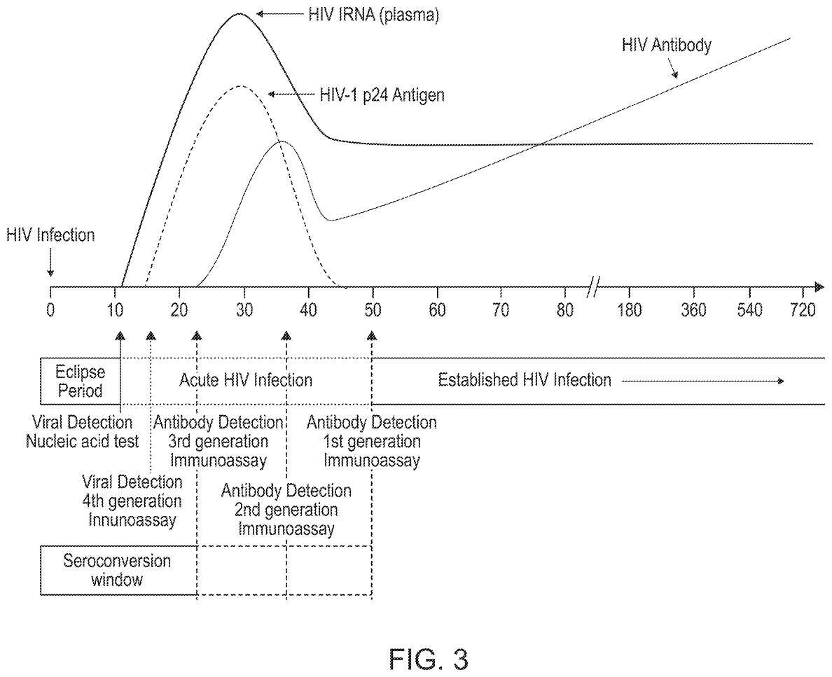

It has been published by the Joint United Nations Programme on HIV/AIDS (UNAIDS) that early detection of HIV can not only have a substantial influence on the success of disease treatment, but it has been shown that individuals in the early stages of the infection have higher transmission rates than later stages. FIG. 3 shows when the screening and differentiation testing can be initiated according to the invasive blood testing methods of the current state of the art. The present invention, however, offers the benefit of noninvasive viral detection at least as early as or earlier than what can be accomplished according to other known methods. This aspect of the invention is critically important in populations such as certain populations in Africa where it is impracticable to carry out diagnoses at the earliest possible stages of infection due to poor transportation infrastructures. The ability of gut cells isolated according to the methods of the present invention allow for longer sample stability even in the absence of refrigeration, enabling the diagnosis of a larger number of patients.

End point testing of cells isolated according to SCSR methods may be at the election of the diagnostic laboratory conducting the tests. The methods described herein comprise isolating, transporting and testing cells from stool samples according to SCSR methods, wherein the test used to detect HIV in cells isolated from the stool sample are selected from the group consisting essentially of antibody tests, viral identification assays, CD4 tests, viral loading tests and early infant diagnostic tests. In certain embodiments, the methods of the present invention may be used to screen a patient population for HIV. In other embodiments, the methods of the present disclosure may also be used to monitor disease progression in patients with HIV. In certain preferred embodiments, broad spectrum GIPSITUMAB are used to detect HIV in infected gut cells isolated as described herein.

In addition to HIV testing, certain embodiments of the present disclosure comprise detection of a biomarker according to a method or process as described herein, wherein the biomarker is a positive or negative indicator of exposure to radiation. In one embodiment of the present invention, the presence of such a biomarker is an indicator of excessive exposure to radiation. In certain embodiments, a biomarker for excessive exposure to radiation may be comprise one or more aberrant proteins detectable by GIPSITUMAB challenged with a specific protein of interest as an antigen.

In one embodiment of the present disclosure, a biomarker detected by a method or process as described herein is an indicator of restoration of neural activity in Hirschsprung's aganglionic segment. This may be achieved by transplantation of healthy gut cells in the affected gut segment of patients suffering from this condition, and in certain embodiments using fecal transplant techniques familiar to one of ordinary skill in the art.

In one embodiment, the SCSR transport medium as described herein may be used for the room temperature preservation of cells from aspirates such as pancreatic, ascites, or pleural effusion aspirates, or for cytopathology.

In one embodiment, a method or process as described herein may be employed for dynamic interrogation of colonic cells for detection of hyperplastic cells as a measure of colon cancer risk. In one embodiment, colon cancer risk may be measured according to such a method or process in patients from families with familial colon cancer syndromes.

In one embodiment, cells isolated according to a method or process of the present disclosure may be used for engraftment of donor colon cells for restorative repair of damage caused by an inflammatory bowel disease (IBD). In one embodiment, the IBD is selected from the group consisting of ulcerative colitis and Crohn's disease.

In one embodiment, cells isolated according to a method or process of the present disclosure may be used for engraftment of donor colon cells expressing RNA-binding protein Musashi homolog 1 (Musashi-1⁺ cells) for counteracting dysbiosis and dyskinesis associated with Parkinson's disease. In one embodiment, such an engraftment may aid in the reversal of the loss of dopaminergic neurons in the substantia nigra.

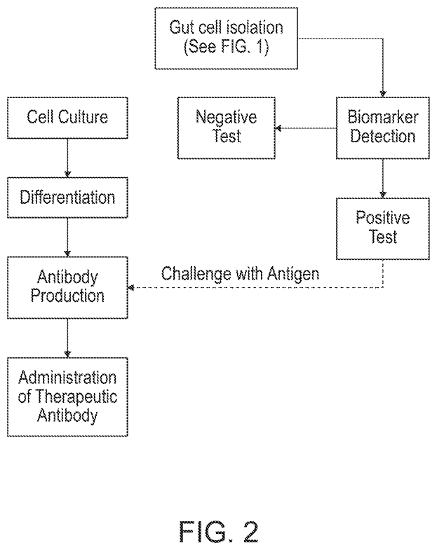

In addition to diagnostic uses, methods of the present invention may be used to grow and differentiate cells in culture which are useful in producing diagnostic and therapeutic antibodies. Turning now to FIG. 2, an exemplary method may begin with gut cell isolation according to FIG. 1, followed by either cell culture or biomarker detection. In the case of biomarker detection, a negative test may be interpreted as an indicator that a patient does not have the disease associated with the biomarker, while a positive test shows the opposite. In the case of cell culture, gut cells may be cultured and then subjected to an agent that differentiates them into antibody-producing GIPC cells, the antibodies so-produced being capable of challenge with known antigens (including those indicative of positive disease diagnosis) for later therapeutic administration to a patient suffering from the disease for which the biomarker is a positive indicator.

These and other embodiments will be apparent to one of ordinary skill in the art. The examples provided herein are not to be construed as limiting the scope of any claim, and are provided by way of illustration rather than limitation.

What is claimed is:

1. A method of isolating antibody-producing cells gut cells, the method comprising:
collecting a fecal sample from a human or animal patient;
preparing a suspension of fecal sample particles in a sterile aqueous solution;
preparing a first filtrate by filtering the suspension to exclude particles that are greater than 330 microns in size;
preparing a second filtrate by filtering the suspension to exclude particles that are greater than 40 microns in size;
overlaying the second filtrate on an underlay cushion and centrifuging at 200 g for ten minutes, wherein centrifuging produces an interface between the cushion and a supernatant;
recovering cells from the supernatant and interface;
preparing a cell suspension by suspending the cells from the supernatant and interface in sterile aqueous solution; and
preparing a pellet by centrifuging the cell suspension for 900 g for ten minutes, wherein the pellet comprises viable antibody-producing gut cells.

2. The method of claim 1, wherein after the step of preparing the cell suspension, the cell suspension may be stored or transported at temperatures of 68 to 78 degrees Fahrenheit for up to five days prior to preparing the pellet.

3. The method of claim 1, further comprising the step of isolating gastrointestinal progenitor stem (GIPC) cells from the pellet, wherein the GIPC cells comprise membrane-bound antibodies.

4. The method of claim 3, further comprising the step of converting the membrane-bound antibodies to chimeric secretory antibodies, each consisting of an immunoglobulin A (IgA) and an immunoglobulin G (IgG) moiety.

5. The method of claim 4, further comprising the step of challenging the chimeric secretory antibodies with an antigen.

6. The method of claim 5, wherein the antigen is a biomarker.

7. The method of claim 6, wherein the biomarker is an indicator of a health condition selected from the group consisting of bacterial infection, viral infection and cancer.

8. The method of claim 5, wherein the health condition is selected from the group consisting of human immunodeficiency virus (HIV) infection, inflammatory bowel disease (IBD), radiation exposure and the restoration of neural activity in Hirschsprung's aganglionic segment.

9. The method of claim 6, wherein the challenged antibodies are rendered responsive to the biomarker (responsive antibodies).

10. The method of claim 9, further comprising the step of propagating the responsive antibodies.

11. A method of diagnosing a health condition, the method comprising:
isolating GIPC cells according to the method of claim 3;
converting the membrane-bound antibodies to chimeric secretory antibodies, each consisting of an IgA and an IgG moiety;
challenging the chimeric secretory antibodies with a biomarker of the health condition to generate responsive antibodies; and
exposing a sample cell population obtained from a patient to the chimeric secretory antibodies, wherein the chimeric secretory antibodies react with cells comprising the biomarker to indicate the presence of the health condition, wherein the sample cell population is obtained by:
collecting a fecal sample from a human or animal patient;
preparing a suspension of fecal sample particles in a sterile aqueous solution;
preparing a first filtrate by filtering the suspension to exclude particles that are greater than 330 microns in size;
preparing a second filtrate by filtering the suspension to exclude particles that are greater than 40 microns in size;
overlaying the second filtrate or an underlay cushion and centrifuging at 200 g for ten minutes, wherein centrifuging produces an interface between the cushion and a supernatant;
recovering cells from the supernatant and interface;
preparing a cell suspension by suspending the cells from the supernatant and interface in sterile aqueous solution; and
preparing a pellet by centrifuging the cell suspension for 900 g for ten minutes.

12. The method of claim 11, wherein the health condition is selected from the group consisting of bacterial infection, viral infection and cancer.

13. The method of claim 11, wherein the health condition is selected from the group consisting of HIV infection, IBD, radiation exposure and the restoration of neural activity in Hirschsprung's aganglionic segment.

14. The method of claim 13, wherein the health condition is HIV infection and the presence of HIV cannot be detected in a blood sample of the patient.

15. A method of producing therapeutic antibodies for the treatment of a health condition, the method comprising:
isolating GIPC cells according to the method of claim 3;
converting the membrane-bound antibodies to chimeric secretory antibodies, each consisting of an IgA and an IgG moiety; and
challenging the chimeric secretory antibodies with a biomarker of the health condition to generate responsive antibodies.

16. A method of treating a health condition, the method comprising producing therapeutic antibodies according to claim 15 and administering an effective dose of the therapeutic antibodies to a patient having the health condition.

17. The method of claim 16, wherein the health condition is selected from the group consisting of bacterial infection, viral infection and cancer.

18. The method of claim 16, wherein the health condition is selected from the group consisting of Ebola virus infection, Marburg virus infection, HIV infection, influenza and anthrax infection.

19. The method of claim 18, wherein the health condition is HIV infection.
